(19) United States
(12) Patent Application Publication
(10) Pub. No.: US 2021/0158940 A1
(43) Pub. Date: May 27, 2021

(54) COMMUNICATION SYSTEM AND METHOD FOR REMOTE SERVICES PLATFORM

(71) Applicant: GLOBAL HEALTH INNOVATORS PTY LTD, Brisbane (AU)

(72) Inventors: Edmund Yee-Lai TSE, Brisbane (AU); Guruparan IYNGKARAN, Brisbane (AU)

(21) Appl. No.: 16/625,642

(22) PCT Filed: Jun. 20, 2018
(86) PCT No.: PCT/AU2018/050611

§ 371 Date: Dec. 20, 2019

(30) Foreign Application Priority Data
Jun. 22, 2017 (AU) 2017902410

(51) Int. Cl. G16H 40/20; G16H 80/00; G16H 15/00

(52) U.S. Cl. CPC G16H 40/20; G16H 15/00; G16H 80/00

(57) ABSTRACT

A communications system for a remote healthcare platform comprises a central server having connected thereto a plurality of electronic devices each operated by a healthcare provider or patient making up a care unit for the patient; and a communications ruleset stored in the central server. The communications ruleset configures the communications system to control the manner in which the plurality of electronic devices communicate with each other, and the communications ruleset is specific to the care unit for the patient.

This application is a National Phase entry of International Application No. PCT/AU2018/050611 under § 371 and claims the benefit of Australian patent application No. 2017902410, filed Jun. 22, 2017, which is hereby incorporated by reference in its entirety.

### FIELD OF INVENTION

The present invention relates to systems and methods for providing communication between users of a remote services platform. The present invention has particular but not exclusive application in the support of remote treatment, care, and management of patients with a chronic disease.

### BACKGROUND OF THE INVENTION

Remote services platforms, such as the remote healthcare system and method disclosed in Australian innovation application number AU2016101455 allow care units comprising of a patient and one or more healthcare providers to be formed. One or more members of a care unit may be remotely located from others of the care unit, and accordingly a means of communication for connecting the members of the care unit is required.

This means of communication may be provided in the form of a conventional electronic messaging protocol, such as email. Email, however, while suited for general communication of a non-specific nature, is ill suited for the specific communication needs in a remote healthcare platform. Email, in particular, is unable to readily provide the following exemplary functions, which, amongst others, are desirable in a remote services platform such as a remote healthcare platform:
Group-to-group messaging where a message is sent as and from a first group of users to a second group of users;
Group-to-individual messaging where a message is sent as and from a first group of users to an individual;
Permission settings for a first individual to allow/disallow individual messaging to/from a second individual;
Permission settings for a first individual to allow/disallow a second individual to view and/or receive all messages sent to the first individual;
Escalation of messages from a first individual to a second individual;
Automatic recording of sent/received messages in patient records;

There is therefore a need for a communications system and method that is capable of providing functions such as those listed above, and others, so as to further build on the efficiency and effectiveness of a remote healthcare platform.

### OBJECT OF THE INVENTION

It is one object of the present invention to provide a communications system and method for forming a messaging map linking a plurality of remote teams together, and realizing communication between remote teams and individuals of each remote team and/or provide the consumer with a useful or commercial choice.

### SUMMARY OF THE INVENTION

According to a first aspect of the present invention, a communications system for a remote healthcare platform comprises a central server having connected thereto a plurality of electronic devices each operated by a healthcare provider or patient making up a care unit for the patient, and a communications ruleset stored in the central server. The communications ruleset configures the communications system to control the manner in which the plurality of electronic devices communicate with each other. The communications ruleset is specific to the care unit for the patient

In one form, the communications system is configured by the communications ruleset to permit a first electronic device to send a message on behalf of one or more other electronic devices.

In one form, the communications system is configured by the communications ruleset to permit a second electronic device to receive a message on behalf of one or more other electronic devices.

In one form, the communications system is configured by the communications ruleset to deny a first electronic device from messaging a second electronic device.

In one form, the communications system is configured by the communications ruleset to have a second electronic device receive a message sent to a first electronic device.

Preferably, the communications system further comprises a messaging map, the messaging map identifying the plurality of electronic devices specific to the care unit and the permitted interactions therebetween. The messaging map further setting the communications ruleset for the care unit.

According to a second aspect of the present invention, a communications method comprises sending a participation request to a selected plurality of participating healthcare providers, selecting one or more personnel from each participating healthcare provider to form a care unit for a patient, registering each selected personnel and an electronic device corresponding to each selected personnel as being part of the care unit, and generating a communications ruleset for configuring messaging permissions within the care unit.

In one form, the participation request is sent to the selected plurality of participants by a central server.

In one form, the method further comprises a step of registering the care unit in the central server, whereby the central server assigns a unit identifier to identify the care unit.

In one form, the one or more personnel are selected by an administrator of each participating provider.

Preferably, the participation request includes one or more of:
Unit identifier
Service requested
Patient details
Treatment/Care/Condition information
Electronic health records
Treatment plans
Preferred/Required health specialists
Preferred/Required health specialist experience/qualifications
Other notes

Preferably, the electronic device corresponding to a selected personnel is sent a notification notifying the selected personnel of their selection.

In one form, the step of setting up the care unit is commenced by a first participant, the first participant selecting the selected plurality of participants to which the participation request are sent.

According to a third aspect of the present invention, a message composition method for a remote healthcare platform comprises selecting, from a finite selection, one or more recipients of the message, composing a body of the message, and sending the message to the selected one or more recipients, wherein the finite selection is determined from a communications ruleset specific to the composer of the message.

### BRIEF DESCRIPTION OF THE DRAWINGS

In order that the present invention can be more readily understood, reference will now be made to the accompanying drawings which illustrate preferred embodiments of the invention and wherein:

FIG. 1 illustrates a communications system according to a first aspect of the present invention;

FIG. 2 further illustrates the communications system according to the first aspect of the present invention;

FIGS. 3 and 4 illustrates an interface for forming a messaging map;

FIG. 5 illustrates a visualization of a messaging map;

FIG. 6 illustrates a method for forming a messaging map;

FIG. 7 illustrates an interface for forming a communications ruleset;

FIG. 8 illustrates an interface for composing a message; and

FIG. 9 illustrates a method for composing a message.

### DETAILED DESCRIPTION OF PREFERRED EMBODIMENTS

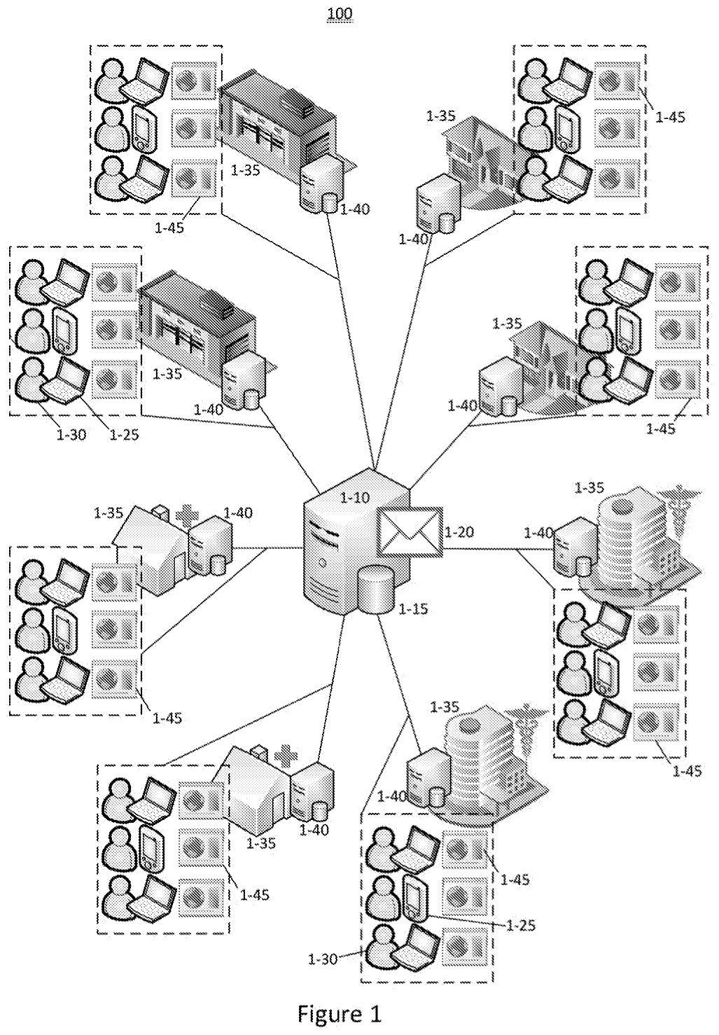

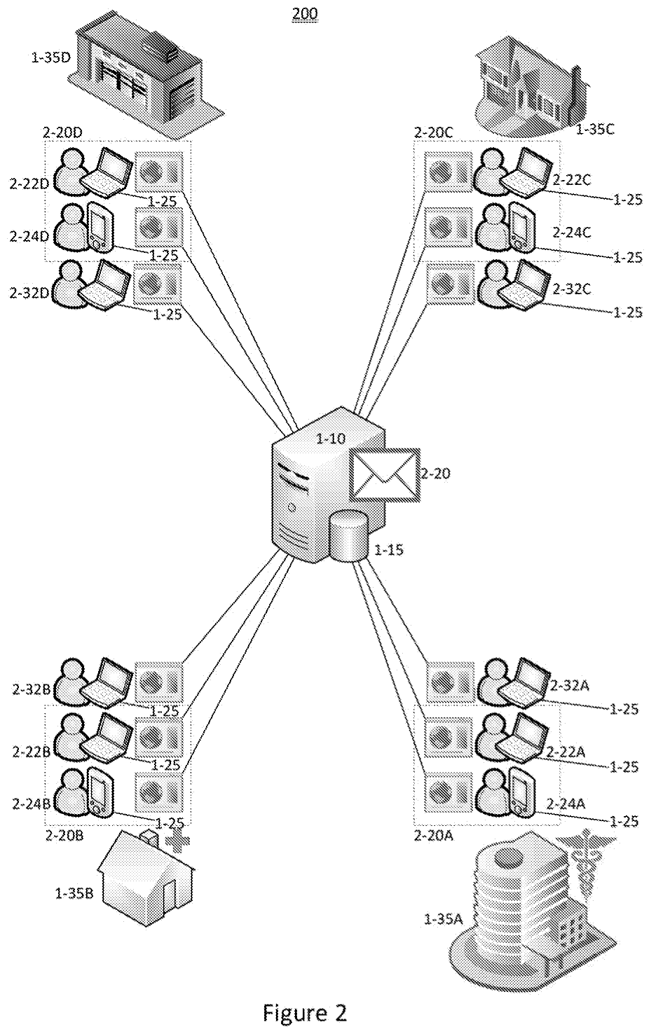

A communications system 100 according to a first aspect of the present invention is illustrated in FIGS. 1 and 2. The communications system 100 is configured for implementation in a remote services platform such as the remote healthcare system described in Australian innovation application number AU2016101455, the contents of which are herein incorporated by reference.

The communications system 100 includes a central server 1-10, a database 1-15, and a messaging control module 1-20. The messaging control module 1-20 is configured and adapted to control and manipulate an operation of the central server 1-10 and/or database 1-15. In a preferred embodiment, the messaging control module 1-20 is realized as software code and/or instructions configured to configure and control the central server 1-10 and/or database 1-15. In another embodiment, the messaging control module 1-20 is realized as a combination of software code and hardware, including logic gates, transistors, memory units, and processing units, and may form and/or include a part of the central server 1-10 and/or database 1-15. In a further embodiment, the messaging control module 1-20 is realized as hardware, comprising transistors and other micro-electronic circuitry.

The communications system 100 further includes a plurality of electronic devices 1-25, each electronically connected to the central server 1-10 via a network, for example the Internet. Each electronic device 1-25 is operated by a user 1-30. Each user 1-30 is associated with a service provider 1-35 such as a healthcare provider (hereinafter, the term healthcare provider may be used interchangeable with the term service provider in the context of the healthcare provider being one example, though not the only example, of a service provider). Each user 1-30 may be associated with the service provider 1-35 as, for example, an employee or a consultant at the service provider 1-35, or, in the case of the service provider 1-35 being a healthcare provider 1-35, as a patient requiring treatment, or as the guardian, carer, or other responsible person of the patient. Each electronic device 1-25 is configured to access a portal 1-45, which in turn connects the electronic device 1-25 to the central server 1-10, database 1-15, and messaging control module 1-20. The portal 1-45 may be a web portal, and/or an application running locally on the electronic device 1-25. The portal 1-25 may include portions of the messaging control module 1-20, such that certain functions provided by the messaging control module 1-20 are performed locally on the electronic device 1-25.

To facilitate clarity, only some users 1-30, electronic devices 1-25, and portals 1-45 have been identified with reference numerals in FIG. 1. However, it is to be understood that all graphics similar to those referenced by reference numeral 1-30 identify a user, all graphics similar to those referenced by reference number 1-25 identify an electronic device, and all graphics similar to those referenced by reference number 1-45 identify a portal.

The invention will now be described by way of an exemplary application of the invention in the field of remote healthcare provision. Specifically, the service provider 1-35 is a healthcare provider, and the users 1-30 are related to or otherwise involved in the provision or receipt of healthcare. It is to be understood, however, that the invention is not limited to application in the field of remote healthcare provision, and may be applicable to any application in which the described control and flexibility of communication provided by the invention is advantageous.

As used herein, a service or healthcare provider 1-35 may be, for example, a hospital, General Practitioner clinic, Specialist Doctor clinic, Allied Health centre (e.g. physiotherapist, traditional Chinese medicine practitioners, etc.), or other establishment involved in the provision of healthcare including, for example, pathology laboratories, radiology laboratories, aged care facilities, hospices, and the like. Each healthcare provider 1-35 may comprise a plurality of users 1-30, and any one user 1-30 may also be associated with multiple healthcare providers 1-35 for example in the case of the user 1-30 being a Specialist Doctor who consults for multiple healthcare providers 1-35.

The communications system 100 may further optionally include local servers 1-40 operated or otherwise managed by each healthcare provider 1-35.

When connected to the central server 1-10, each electronic device 1-25 connects as a representative of one of the healthcare providers 1-35. Specifically, and as will be described in greater detail below with reference to FIG. 2, each electronic device 1-25 when connected to the central server 1-10 identifies itself as a member of the healthcare provider 1-35 that its user 1-30 is associated with. In the case of the electronic device 1-25 that is used by the patient, the electronic device 1-25 identifies itself as a member of the patient's support team. In the case of the user 1-30 of an electronic device 1-25 being associate with more than one healthcare provider 1-35, the electronic device 1-25 identifies to the central server 1-10 as a member of the healthcare provider 1-35 that its user is currently rendering service on behalf of.

FIG. 2 illustrates a composition of a care unit 200. As illustrated in FIG. 2, a care unit 200 for a patient 2-22C is made up of a number of health teams 2-20A, 2-20B, 2-20C, 2-20D. Each health team 2-20A, 2-20B, 2-20C, 2-20D is made up of one or more users 1-30 from respective healthcare providers 1-35A, 1-35B, 1-35C, 1-35D. An exemplary care unit 200 as shown in FIG. 2 is made up of, for example:
Specialist Doctor 2-22A from hospital 1-35A, as part of Specialist health team 2-20A;
Specialist Nurse 2-24A from hospital 1-35A, as part of Specialist health team 2-20A;
General Practitioner (GP) Doctor 2-22B from GP Clinic 1-35B, as part of GP health team 2-20B;
GP Nurse 2-24B from GP Clinic 1-35B, as part of GP health team 2-20B;
Patient 2-22C living in home 1-35C, as part of patient support team 2-20C;
Carer 2-24C living/working in home 1-35C, as part of patient support team 2-20C (or alternatively, as part of a separate hospice healthcare provider);
Physiotherapist 2-22D from medical centre 1-35D, as part of allied health support team 2-20D; and
Dietician 2-24D from medical centre 1-35D, as part of allied health support team 2-20D

The members of each health team 2-20A, 2-20B, 2-20C, 2-20D are determined by practice administrators 2-32A, 2-32B, 2-32C, 2-32D. As will be described in greater detail below, each practice administrator 2-32A, 2-32B, 2-32C, 2-32D forms a health team 2-20A, 2-20B, 2-20C, 2-20D in response to their respective healthcare providers 1-35A, 1-35B, 1-35C, 1-35D receiving an invitation to join a specific care unit 200. In accordance with the present invention, each electronic device 1-25 of each user 1-30 of each health team 2-20A, 2-20B, 2-20C, 2-20D, as well as the combination of electronic devices 1-25 in each health team 2-20A, 2-20B, 2-20C, 2-20D as a single collective entity, together with the central server 1-10, database 1-15, and messaging control module 1-20 form a care unit messaging map specific to the care unit 200.

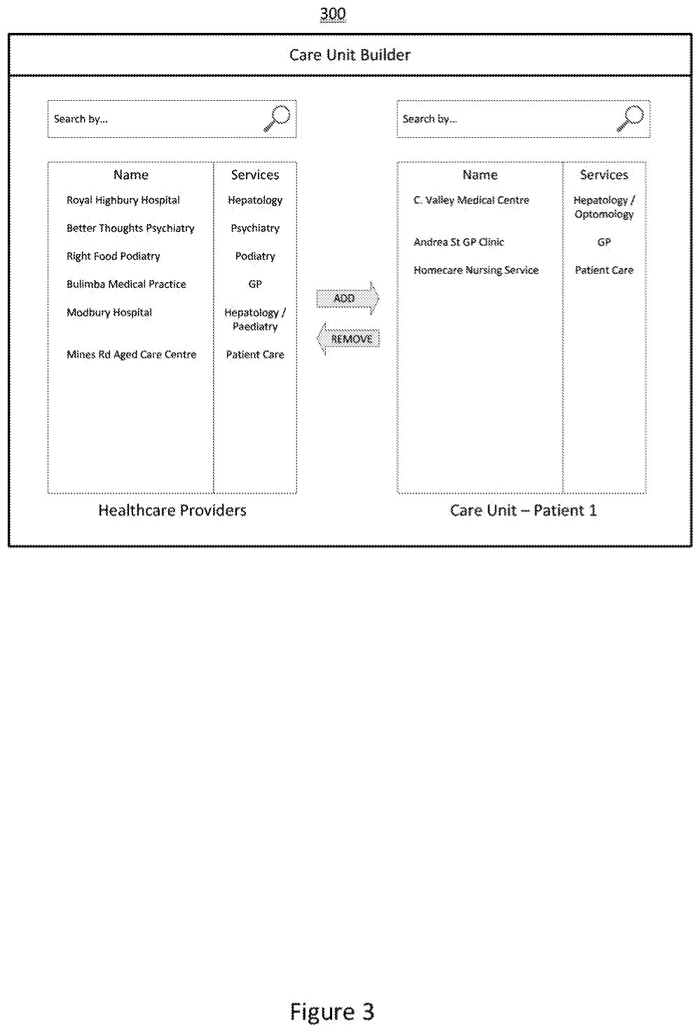

With reference now to FIGS. 3 to 6, a process and method 600 for forming a messaging map for a care unit 200 is described.

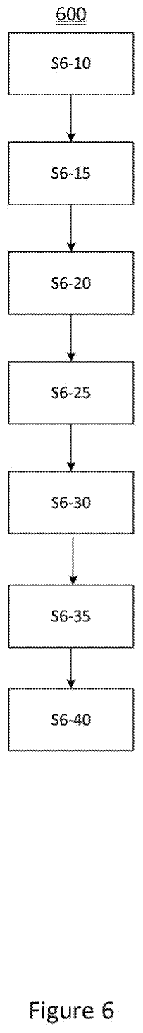

The method 600 commences at S6-10 (FIG. 6), where a health care provider 1-35, for example the GP Clinic 1-35B to which a patient 2-22C first presents, makes a decision to create a care unit 200 to attend to the treatment and care of the patient 2-22C. To commence the process for creating the care unit 200, the electronic device 1-25 of the practice administrator 2-32 for the health unit 2-30 (in this example, the practice administrator 2-32B of the GP Clinic 1-35B) generates and submits to the central server 1-10 a request to create a new care unit 200. Upon receipt of the request, the central server 1-10 creates a care unit identifier that uniquely identifies the newly created care unit.

At S6-15, the electronic device 1-25 operated by the practice administrator 2-32 accesses the database 1-15 to search for health care providers. The search for health care providers may be by specified criteria, or by browsing all available providers. For example, the criteria may specify one or more of a service provided, a geographic location, a specific professional, and the like. Results of the search are displayed in a care unit builder 300 (FIG. 3), which is a webpage or other portal interface generated by the central server 1-10, local server 1-40, and/or electronic device 1-25, and presented to the practice administrator 2-32 via a respective portal 1-45.

At S6-20, the practice administrator 2-32, by way of the care unit builder 300, selects one or more health care providers to comprise the care unit 200. In the example illustrated by FIG. 3, the practice administrator 2-32 selects “C. Valley Medical Centre” as a hepatology/optomology healthcare provider, “Andrea St GP Clinic” as the GP service provider, and “Homecare Nursing Service” as the patient care service provider.

At S6-25, once the healthcare providers 1-35 desired to comprise the care unit 200 have been selected, a care unit participation request is generated and sent out from the central server 1-10 to each of the selected healthcare providers 1-35 (also referred to as participating providers), and preferably, to the respective practice administrators 2-32 of each healthcare provider 1-35 and/or respective local server 1-40. In the preferred embodiment, the participation request includes one or more of:
Care unit identifier
Service requested
Patient details
Treatment/Care/Condition information
Electronic health records
Treatment plans
Preferred/Required health specialists
Preferred/Required health specialist experience/qualifications
Other notes

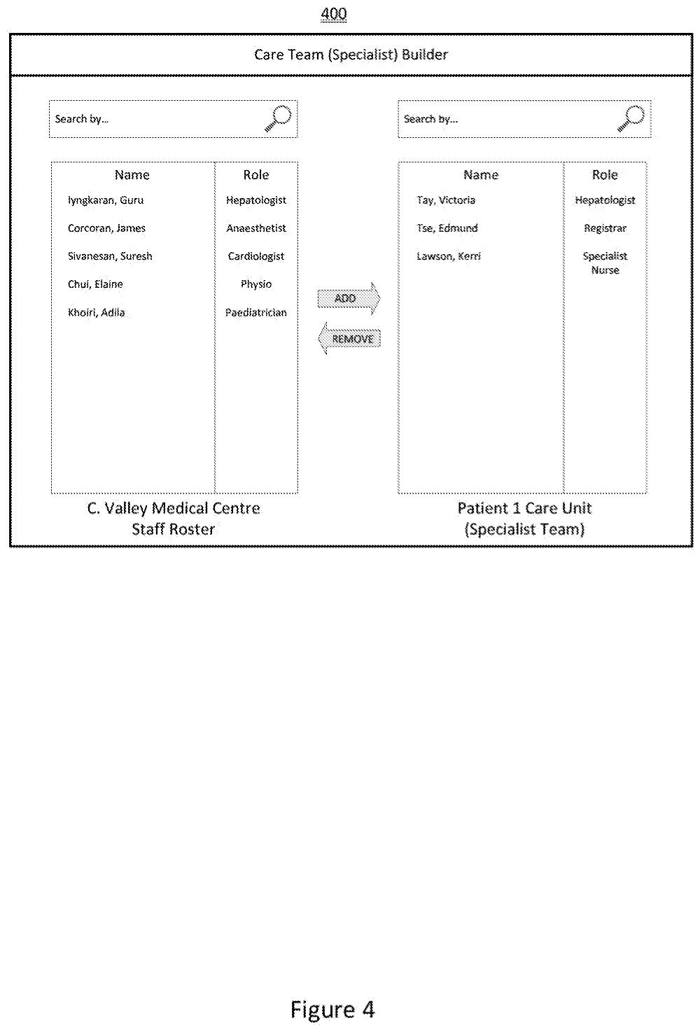

At S6-30, upon receipt of the participation request, each healthcare provider 1-35 builds a health team 2-20 to satisfy the participation request. The building of the health team 2-20 is effected by way of a care team builder 400 (FIG. 4). The care team builder 400 is, for example, a webpage or other portal interface listing valid personnel (e.g. personnel registered with the communications system 100) for inclusion in the health team 2-20. The care team builder 400 may be generated by the central server 1-10, the local server 1-40 of the healthcare provider 1-35, and/or the electronic device 1-25 of the practice administrator 2-32.

At S6-35, each personnel added to the health team 2-20 is notified by a message sent to respective electronic devices 1-25 operated thereby.

At S6-40, the individual personnel comprising each health team 2-20, and each health team 2-20, are registered in the central server 1-10 as the care unit 200. Additionally, the electronic devices 1-25 belonging to each personnel of the care unit 200 are registered as part of a messaging map specific to the care unit 200.

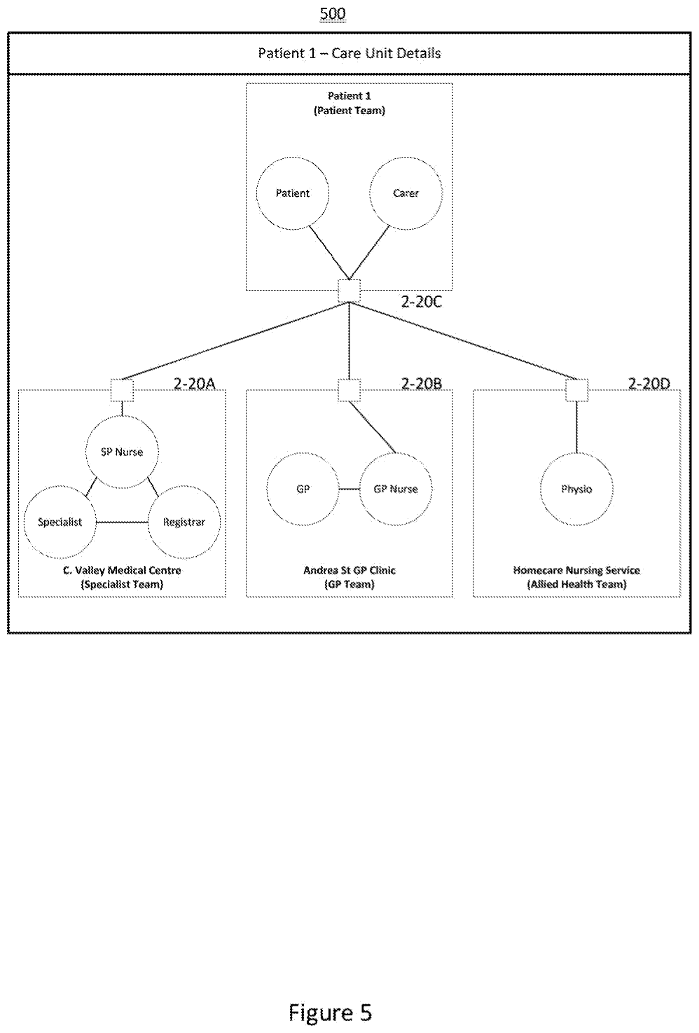

For purposes of convenient description, the messaging map is illustrated as a visualization 500 (FIG. 5). The messaging map can outline and/or set a a communications ruleset controlling communication between the health teams 2-20 and between the personnel (i.e. users 1-30) in each health team 2-20, and can also facilitate modification of the communications ruleset and/or generation of a digital file representing the communications ruleset that can then be read/executed by the central server 1-10 and electronic devices 1-25 as appropriate. It is to be understood that the present invention is however not so limited, and that other methods may be used to set, outline, modify, and/or generate the communications ruleset. The invention is also not limited to the messaging map being illustrated as the visualization 500.

As illustrated by the visualization 500, the newly created care unit 200 is set up with an exemplary default communications ruleset allowing a nominated member of each health team 2-20 to send and receive messages to other health teams 2-20, and for each nominated member to further disseminate received messages to specific members of their health teams 2-20. In this exemplary default communications ruleset, messaging from an individual member 1-30 of a health team 2-20 to an individual member 1-30 of another health team 2-20 is not permitted, nor is messaging from an individual member 1-30 to a health team 2-20. The default communications ruleset can, however, be modified to permit any combination of individual to individual, individual to health team, and health team to individual messaging. For example, in a preferred default communications ruleset, all messages received to a health team 2-20 are automatically disseminated to all members 1-30 of the health team 2-20. Similarly, the sending of a message by a member 1-30 of a health team 2-20 is automatically sent as if from the entire health team 2-20.

In other embodiments, the communications ruleset may be set such that the GP Doctor 2-22B can individually message the Patient 2-22C, and the individual message from the GP Doctor 2-22B to the Patient 2-22C made confidential and inaccessible to anyone but the Patient 2-22C. The ruleset can also be configured such that return messages from the Patient 2-22C to the GP Doctor 2-22B, and indeed any messages sent individually to the GP Doctor 2-22B, are automatically disseminated or otherwise made accessible to the GP Nurse 2-24B.

In one embodiment, modifications to the communications ruleset can be made by individual members 1-30 of each health team 2-20, a nominated member 1-30 of the health team 2-20 on behalf of another member 1-30, and/or by the practice administrator 1-32. In another embodiment, different portions of the communications ruleset may be made modifiable by different members 1-30. For example, one or more members 1-30 of the Specialist health team 2-20A may be permitted to modify the communications ruleset as it pertains to the Specialist health team 2-20A, but not to other health teams 2-20.

In the preferred embodiment, the communications ruleset is stored in the central server 1-10, and local copies thereof may also be stored (and updated as required) on the local servers 1-40 of each healthcare provider 1-35, and/or on individual electronic devices 1-25. As will be described in greater detail below, the communications ruleset is accessed and referenced by the electronic devices 1-25 and central server 1-10 prior to and/or during the conduct of various functions such as the composing of messages, sending of messages, receiving of messages, and the like.

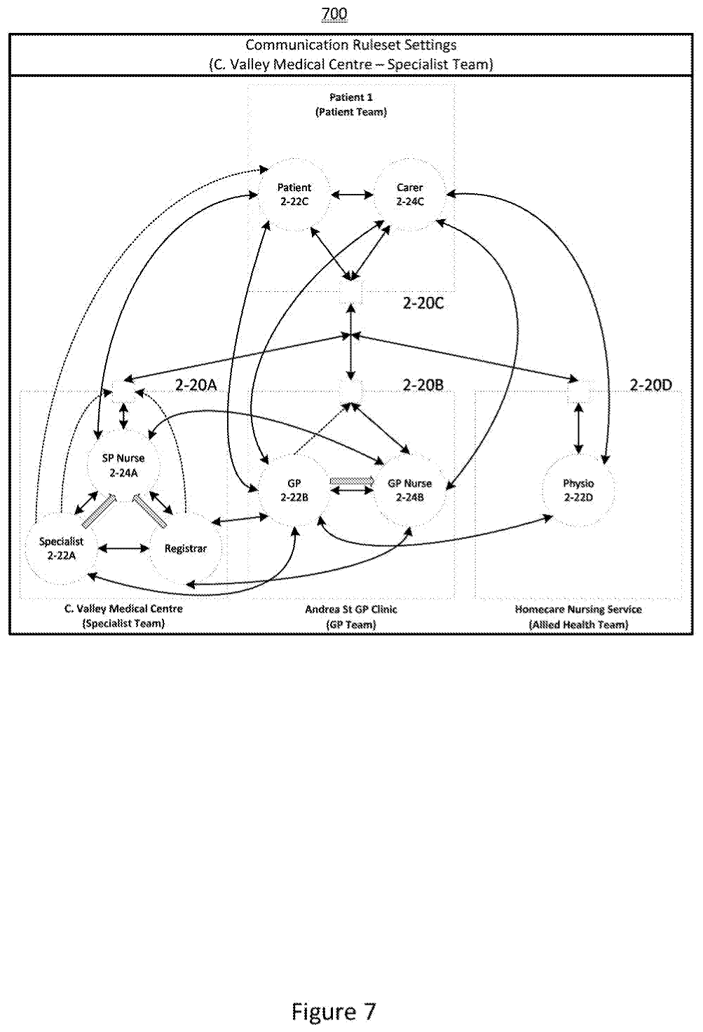

FIG. 7 illustrates a communications ruleset setting interface 700. In the preferred embodiment, the communications ruleset setting interface 700 is dynamically generated and populated each time it is loaded and accessed with the communications ruleset from the central server 1-10 and the current members of the care unit 200. In other embodiments, a local copy of the communications ruleset may be stored locally on the electronic devices 1-25 and/or local servers 1-40, and updated with the central server 1-10 periodically and/or as needed.

The communications ruleset setting interface 700 illustrated in FIG. 7 is a visual interface, wherein a line connecting two parties indicates that a message channel exists between the two parties, and direction of the line as indicated by the arrowhead indicates the direction in which messaging between the two parties is permitted. It should be understood that while the interface 700 illustrated in FIG. 7 is a visual interface, the present invention is not so limited and communications ruleset settings may also be set by way of a tradition text interface such as through the use of menus, radio buttons, command-line interfaces, and the like.

According to the exemplary communications ruleset illustrated in FIG. 7:

General
Each health team 2-20 can directly send to and receive messages from any other health team 2-20, as indicated by double headed arrows joining each health team 2-20

Specialist Health Team 2-20A
Messages sent to the Specialist health team 2-20A are received only by the Specialist Nurse 2-24A.
Each of the Specialist Doctor 2-22A, Registrar, and Specialist Nurse 2-24A can send messages as the Specialist health team 2-20A;
All members of the Specialist health team 2-20A can send and receive messages directly to each other;
The Specialist Doctor 2-22A can send messages directly to the Patient 2-22C, but the Patient 2-22C cannot reply directly to the Specialist Doctor 2-22A;
The Specialist Nurse 2-24A can send messages directly to the Patient 2-22C, and the Patient can reply directly to the Specialist Nurse 2-24A;
The Registrar can send messages directly to the GP Doctor 2-22B, and the GP Doctor 2-22B can reply directly to the Registrar;
The Registrar can send messages directly to the GP Nurse 2-24B, and the GP Nurse 2-24B can reply directly to the Registrar; and
The Specialist Nurse 2-24A can send messages directly to the GP Nurse 2-24B, and the GP Nurse 2-24B can reply directly to the Specialist Nurse 2-24A.

GP Health Team 2-20B
Messages sent to the GP health team 2-20B are received only by the GP Doctor 2-24B;
Both the GP Doctor 2-22B and the GP Nurse 2-24B can send messages as the GP health team 2-20B;
Both members of the GP health team 2-20B can send and receive messages directly to each other;
The GP Doctor 2-22B can send messages directly to the Specialist 2-22A, and the Specialist 2-22A can reply directly to the GP Doctor 2-22B;
The GP Doctor 2-22B can send messages directly to the Registrar, and the Registrar can reply directly to the GP Doctor 2-22B;
The GP Doctor 2-22B can send directly messages directly to the Physio 2-22D, and the Physio 2-22D can reply directly to the GP Doctor 2-22B;
The GP Doctor 2-22B can send messages directly to the Patient 2-22C, and the Patient 2-22C can reply directly to the GP Doctor 2-22B;
The GP Nurse 2-24B can send messages directly to the Specialist Nurse 2-24A, and the Specialist Nurse 2-24A can reply directly to the GP Nurse 2-24B; and
The GP Nurse 2-24B can send messages directly to the Registrar, and the Registrar can reply directly to the GP Nurse 2-24B.

Allied Health Team 2-20D
Messages sent to the Allied Health Team 2-20D are received only by the Physio 2-22D;
The Physio 2-22D can send messages as the Allied Health Team 2-20D
The Physio 2-22D can send messages directly to the Registrar, and the Registrar can reply directly to the Physio 2-24D; and
The Physio 2-22D can send messages directly to the Carer 2-24C, and the Carer 2-24C can reply directly to the Physio 2-24D.

Patient Support Team 2-20C
Messages sent to the Patient Support Team 2-20C are received by both the Patient 2-22C and the Carer 2-24C;
Both members of the Patient Support Team 2-20C can directly message each other;
Both members of the Patient Support Team 2-20C can send messages as the Patient Support Team 2-20C;
The Patient 2-22C can receive messages directly from the Specialist Doctor 2-22A but cannot directly reply to the Specialist Doctor 2-22A;
The Patient 2-22C can send messages directly to the Specialist Nurse 2-24A, and the Specialist Nurse 2-24A can directly reply to the Patient 2-22C;
The Patient 2-22C can send messages directly to the GP Doctor 2-22B, and the GP Doctor 2-22B can directly reply to the Patient 2-22C;
The Carer 2-24C can send messages directly to the GP Nurse 2-24B, and the GP Nurse 2-24B can reply directly to the Carer 2-24C;
The Carer 2-24C can send messages directly to the Physio 2-22D, and the Physio 2-22D can reply directly to the Carer 2-24C.

The above communications ruleset as illustrated by the communications ruleset setting interface 700 of FIG. 7 is to be understood as exemplary only.

The communications ruleset setting interface 700 further allows access permissions to be set. Access permissions dictate which members, if any, can access the messages of another member and are indicated by solid 3D arrows. According to the exemplary communications ruleset illustrated in FIG. 7 the Specialist Nurse 2-24A has permission to access the messages of the Specialist Doctor 2-22A and the Registrar. Similarly, the GP Nurse 2-24B has permission to access the messages of the GP Doctor 2-22B.

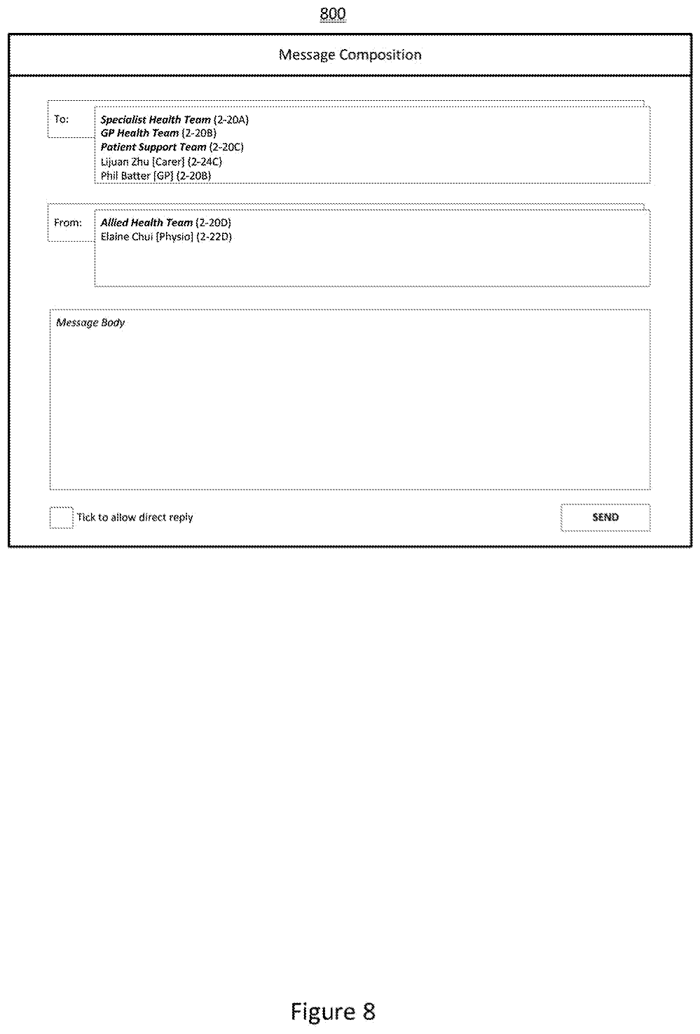

FIG. 8 illustrates a message composition interface 800 according to the present invention. The message composition interface 800 comprises a “To” field 8-10, a “From” field 8-20, a “Message body” field 8-30, a direct reply permission setting 8-40, and a send button 8-50.

According to the preferred embodiment, the “To” field 8-10 is populated only with valid recipients to which the sender can send a message, as set by the communications ruleset. The list of valid recipients for the “To” field 8-10 can be updated live from the central server 1-10 each time a message is composed, or can be generated from a locally stored copy of the communications ruleset. In one embodiment, the “To” field 8-10 does not permit free-text entry. Similarly, the “From” field 8-20 is populated only with valid identities from which the sender can send a message, as set by the communications ruleset. As with the “To” field 8-10, the “From” field 8-20 can be updated live from the central server 1-10 each time a message is composed, or can be generated from a locally stored copy of the communications ruleset. The “Message body” field 8-30 is configured to receive free text that makes up the message, and the send button 8-50 sends the message to the identified recipients. The direct reply permission setting 8-40 provides an alternative manner for the sender of the message to change the communications ruleset. In particular, it provides a quick and convenient method for the sender to revise the communications ruleset to allow all recipients of the message to be able to reply directly to the sender. In one embodiment, the direct reply permission setting 8-40 temporarily changes the communications ruleset to allow a once-of direct reply to the sender of the message.

While not illustrated, it is to be understood that the message composition interface 800, and the messages of the communications system 100, allow for other functions that are common to messaging protocols, including attachments, read receipts, forwarding, delayed sending, priority indications, and the like.

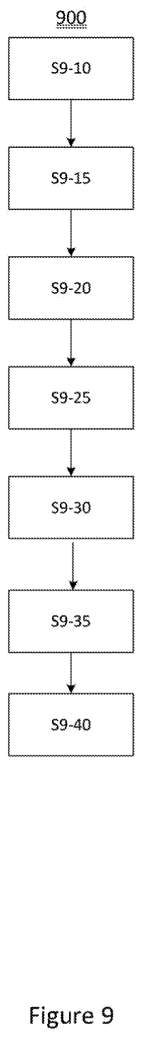

With reference to FIG. 9, a process and method 900 for composing a message is described.

At S9-10, generation of the message composition interface 800 is commenced in response to an operation of an electronic device 1-25 by a user 1-30 to compose a message. The message composition interface 800 is generated by one or more of the central server 1-10 or electronic device 1-25, either wholly or partially, and presented to the user 1-30 via the portal 1-45.

At S9-15, as part of the generation of the message composition interface 800, a communications ruleset is accessed. The communications ruleset may be stored locally on the electronic device 1-25 and thereby accessed locally, or stored remotely on a server (for example the central server 1-10, and/or local server 1-40) and accessed remotely over a network.

At S9-20, a “To” field 8-10 of the message composition interface 800 is populated in accordance with the accessed communications ruleset. Specifically, only recipients that the user commencing the message composition operation is allowed to message, as specified by the communications ruleset, are populated into the available options of the “To” field 8-10.

Similarly, at S9-25, the “From” field 8-20 of the message composition interface 800 is populated in accordance with the accessed communications ruleset. Specifically, only identities that the user commencing the message composition operation is allowed to send messages as, as specified by the communications ruleset, are populated into the available options of the “From” field 8-20.

At S9-30, the user 1-30 composes a message in the “Message body” field 8-30. The message may be composed as plain text, HTML formatted text, or other formats, and may include attachments, in-line graphics, embedded files, and the like.

At S9-35, the user 1-30 toggles the direct reply permission setting 8-40 as appropriate/desired.

At S9-40, the user 1-30 operates the send button 8-40 to send the composed message to the recipients identified in the “From” field 8-10.

At S9-45, each recipient of the messages is notified of the message that was sent at S9-40.

In a preferred embodiment of the communications system 100, messages sent and received by the various users 1-30 may be automatically saved to an electronic record, for example a patient's Electronic Health Record (EHR), doctor's patient notes, and the like. The communications system 100 may be configured to automatically save all messages, manually save only specified messages, automatically save all messages matching a specified criteria, and the like.

### ADVANTAGES

The present invention provides a communications system and method that is particularly suited for messaging within a remote healthcare platform. The system and method of the present invention provides greater control, flexibility, and options compared to that of existing electronic messaging systems such as email. Moreover, the system and method of the present invention allows for the creation of multiple, separate, closed-group messaging maps that link specific entities together, each with a bespoke and revisable communications ruleset that is tailored to the group's needs.

### VARIATIONS

It will of course be realised that while the foregoing has been given by way of illustrative example of this invention, all such and other modifications and variations thereto as would be apparent to persons skilled in the art are deemed to fall within the broad scope and ambit of this invention as is herein set forth.

Throughout the description and claims of this specification the word “comprise” and variations of that word such as “comprises” and “comprising”, are not intended to exclude other additives, components, integers or steps.

1-15. (canceled)

16. A communications system for a remote healthcare platform, the communications system comprising:
a central server having connected thereto a plurality of electronic devices each operated by a healthcare provider or patient making up a care unit for the patient; and
a communications ruleset stored in the central server, wherein
the communications ruleset configures the communications system to control the manner in which the plurality of electronic devices communicate with each other, and
the communications ruleset is specific to the care unit for the patient.

17. A communications system as claimed in claim 16, wherein the communications system is configured by the communications ruleset to permit a first electronic device to send a message on behalf of one or more other electronic devices.

18. A communications system as claimed in claim 16, wherein the communications system is configured by the communications ruleset to permit a second electronic device to receive a message on behalf of one or more other electronic devices.

19. A communications system as claimed in claim 16, wherein the communications system is configured by the communications ruleset to deny a first electronic device from messaging a second electronic device.

20. A communications system as claimed in claim 16, wherein the communications system is configured by the communications ruleset to have a second electronic device receive a message sent to a first electronic device.

21. A communications system as claimed in claim 16, wherein the communications system further comprises a messaging map that identifies the plurality of electronic devices specific to the care unit and the permitted interactions therebetween, the messaging map setting the communications ruleset for the care unit.

22. A communications method for a remote healthcare platform, the method comprising:
sending a participation request to a selected plurality of participating healthcare providers;
selecting one or more personnel from each participating healthcare provider to form a care unit for a patient;
registering each selected personnel and an electronic device corresponding to each selected personnel as being part of the care unit, and
generating a communications ruleset for configuring messaging permissions within the care unit.

23. A communications method as claimed in claim 22, wherein the participation request is sent to the selected plurality of participating healthcare providers by a central server.

24. A communications method as claimed in claim 22, further comprising a step of registering the care unit in the central server, whereby the central server assigns a unit identifier to identify the care unit.

25. A communications method as claimed in claim 22, wherein the one or more personnel are selected by an administrator of each participating healthcare provider.

26. A communications method as claimed in claim 22, wherein the participation request includes one or more of:
Unit identifier
Service requested
Patient details
Treatment/Care/Condition information
Electronic health records
Treatment plans
Preferred/Required health specialists
Preferred/Required health specialist experience/qualifications
Other notes.

27. A communications method as claimed in claim 22, wherein the electronic device corresponding to a selected personnel is sent a notification notifying the selected personnel of their selection.

28. A communications method as claimed in claim 22, wherein the step of setting up the care unit is commenced by a first participant, the first participant selecting the selected plurality of participants to which the participation request are sent.

29. A message composition method for a remote healthcare platform, the method comprising:
selecting, from a finite selection, one or more recipients of the message;
composing a body of the message; and
sending the message to the selected one or more recipients, wherein
the finite selection is determined from a communications ruleset specific to the composer of the message.
